(19) United States
(12) Patent Application Publication
(10) Pub. No.: US 2025/0262448 A1
(43) Pub. Date: Aug. 21, 2025

(54) MINIMALLY INVASIVE MINI-COIL MAGNETIC NEURAL STIMULATOR

(71) Applicant: William Marsh Rice University, Houston, TX (US)

(72) Inventors: Taiyun Chi, Houston, TX (US); Chong Xie, Houston, TX (US); Yingying Fan, Houston, TX (US); Yuxuan Liu, Houston, TX (US)

(73) Assignee: William Marsh Rice University, Houston, TX (US)

(21) Appl. No.: 19/049,958

(22) Filed: Feb. 10, 2025

Related U.S. Application Data
(60) Provisional application No. 63/554,673, filed on Feb. 16, 2024.

(51) Int. Cl. A61N 2/02; A61N 2/00

(52) U.S. Cl. CPC A61N 2/02; A61N 2/006

(57) ABSTRACT

A mini-coil magnetic neural stimulator (MCMS) includes a coil that generates magnetic stimulation and a coil driver circuit that controls the magnetic stimulation. The coil driver circuit may include an H-bridge gate driver that includes a plurality of switches that are arranged in the shape of an “H;” a storage capacitor that stores energy that is used to power the coil during the magnetic stimulation. The coil driver circuit may further include a gate driver that controls switching of the plurality of switches. The coil driver circuit may further include a timing signal generation module that generates a first pulse generator a deadtime controller, a second pulse generator, and a zero-current switching controller.

### CROSS-REFERENCE TO RELATED APPLICATIONS

This application claims priority to and the benefit of the filing date of U.S. Provisional Patent Application 63/554,673, filed on Feb. 16, 2024, which is hereby incorporated by reference herein in its entirety.

### BACKGROUND

Neurological disorders pose significant challenges, often requiring invasive treatments such as intracortical microstimulation (ICMS) or deep brain stimulation (DBS). Conventionally, stimulation is delivered by implanted electrodes, such as in ICMS, and DBS. However, long-term reliability associated with the electrode-tissue interface remains a fundamental challenge, due to foreign body responses that can lead to glial scar formation and encapsulation of electrodes. Magnetic stimulation has emerged as a promising alternative to overcome this challenge, as no direct contact is needed between the stimulation coil and tissues. Its efficacy has been clinically verified through FDA-approved transcranial magnetic stimulation (TMS) for treating neurological disorders. However, TMS devices are power-hungry and bulky, which not only restricts patient accessibility but also limits the spatial resolution of the stimulation.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, one or more embodiments of the invention are directed to a mini-coil magnetic neural stimulator (MCMS) that includes a coil for magnetic stimulation and a coil driver circuit. The coil driver circuit may include an H-bridge power stage, in which the switches are arranged in the shape of an “H.” The coil driver circuit may further include an off-chip storage capacitor. The coil driver circuit may further include an H-bridge gate driver with a gate driver that controls the switching of the transistors in the H-bridge power stage. The coil driver circuit may further include a timing signal generation module that includes a first pulse generator, a deadtime (DT) controller, a second pulse generator, a level shifter, and a zero-current switching (ZCS) controller.

In general, in one aspect, one or more embodiments of the invention are directed to a method of treating neurological disorders. The method includes implanting the MCMS. The MCMS may include a coil for magnetic stimulation and a coil driver circuit in a skull burr hole without penetrating a dura of a patient. No external leads are connected to the MCMS through the scalp.

Other aspects and advantages of one or more embodiments disclosed herein will be apparent from the following description and the appended claims.

### BRIEF DESCRIPTION OF DRAWINGS

FIGS. 1A-1C illustrate the proposed mini-coil magnetic stimulator (MCMS) sealed within a burr hole in accordance with various embodiments;

FIGS. 2A-2C show a coil-circuit co-optimization flow in accordance with various embodiments;

FIG. 3 is a schematic drawing in accordance with various embodiments;

FIGS. 4A-4B are schematic drawings in accordance with various embodiments;

FIGS. 5A-5D show measured waveforms in accordance with various embodiments;

FIGS. 6A-6B describe an experimental setup in accordance with various embodiments;

FIG. 7 shows the testing PCB and chip micrograph in accordance with various embodiments;

FIG. 8 illustrates an experiment setup in accordance with various embodiments;

FIGS. 9A-9D show a measurement result in accordance with various embodiments.

### DETAILED DESCRIPTION

Specific embodiments of the invention will now be described in detail with reference to the accompanying figures.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

The Magnetic Stimulator (MCMS), featuring a miniaturized coil and a compact, programmable, and energy-efficient driver chip is a minimally invasive, energy-efficient solution for magnetic brain stimulation. The MCMS is implanted in a skull burr hole and reduces risks associated with traditional methods. Its small coil diameter and custom-designed CMOS driver chip allow precise, low-power neural stimulation. With successful demonstrations in mice, ongoing research aims to optimize stimulation parameters, making MCMS a promising advancement for non-invasive neuromodulation in clinical and research applications. Embodiments of the MCMS device include the following highlights.

In one or more embodiments, the MCMS device is a magnetic neural stimulator sitting in the skull burr hole, targeting human cortex stimulation without penetrating the dura. It imposes considerably lower risk and invasiveness than implanting intra-cortical electrodes or micro-coils into the brain tissue. The MCMS may include a coil for magnetic stimulation and a coil driver circuit in a skull burr hole without penetrating a dura of a patient. No external leads are connected to the MCMS through the scalp.

In one or more embodiments, an optimization flow is used to determine the optimal coil geometry while ensuring effective neural stimulation with the lowest power consumption under the device size constraints (i.e., fit within a skull burr hole).

In one or more embodiments, a custom-designed driver chip in CMOS is used for high energy efficiency. The chip uses an H-bridge driver topology that outputs a substantially triangular stimulation waveform with programmable amplitude, pulse duration, and duty cycle. Along with high-efficiency circuit design techniques such as ZCS control and deadtime control, MCMS achieves the highest charge recovery efficiency (99.76%) and the lowest DC power consumption (≤12 mW) among reported magnetic neural stimulators.

In one or more embodiments, a coil driver circuit is an electronic circuit that controls the direction and magnitude of current flow across a load, typically a motor or in this case, a coil in the MCMS. The coil driver circuit includes an H-bridge configuration that includes four switches arranged in the shape of the letter “H.” These switches can be transistors (e.g., MOSFETs or IGBTs) or other semiconductor devices. The H-bridge allows for bidirectional current flow, enabling the coil to generate magnetic fields with alternating polarity. This capability is essential for neural stimulation applications where precise control of the stimulation waveform is required.

In the context of the MCMS, the coil driver circuit is utilized to control the current flowing through the miniaturized coil. The programmable nature of the coil driver circuit in the MCMS enables the generation of a substantially triangular stimulation waveform with customizable parameters. This waveform is then applied to the coil, allowing for targeted and efficient neural stimulation in a controlled manner.

The MCMS's stimulation capability is validated in vivo by stimulating the mouse brain transcranially and analyzing the brain activity using wide-field Ca²⁺ fluorescence imaging.

MCMS can be potentially scaled into an array of stimulators, where each individual coil can be digitally programmed. This allows superior resolution of the stimulation and can cover a network of neurons for multi-site stimulation.

In general, an aspect of embodiments of the invention is directed to a device that is a minimally invasive MCMS. The MCMS device features a design that is 0.5 cm to 1.5 cm in diameter, 0.5 cm in height, requires less than 12 mW of power. The MCMS device is implanted in a patient's skull burr. This device MCMS provides an optimized and more economical option to Transcranial magnetic stimulation (TMS), which is used to stimulate nerve cells in the brain. Potential applications include treating depression, anxiety, obsessive-compulsive disorder (OCD), or motor dysfunction.

The miniaturized design of MCMS makes it capable of implantation through the skull and, together with the novel, energy-efficient design of the chip, for portable or even wireless powering. It can cover almost all existing application scenes of traditional transcranial magnetic stimulation (TMS) while doing things in a more convenient and accurate way.

The MCMS system can be applied to substantially all clinical scenarios where traditional Transcranial Magnetic Stimulation (TMS) is utilized, offering even greater convenience and stimulation accuracy. TMS, which has received FDA approval, has demonstrated efficacy in treating a spectrum of neural disorders, including migraine, stroke rehabilitation, neuropathic pain, depression, bipolar disorder, and more. With its ability to be implanted and superior focality all coming from being miniaturized, the MCMS system will significantly advance current understanding of the fundamental mechanisms underlying neural disorders and facilitate tailored therapeutic interventions.

The MCMS system introduces novel implantable neuromodulation methods that enhance resolution and energy efficiency in animal laboratory research. Its compact size, energy-saving features, and user-friendly interface make it particularly well-suited for researchers in academic institutions and companies. This system empowers researchers to deliver highly selective and customizable stimulation to different brain regions while minimizing potential damage to brain tissue in animals.

In one or more embodiments, the MCMS may be minimally invasive, consisting of a cm-scale coil and a programmable driver chip. In one or more embodiments, the MCMS may be viewed as a “miniaturized transcranial magnetic stimulation (TMS) device” but implanted in a skull burr hole. Compared with traditional TMS, however, the coil diameter is reduced from 10-20 cm in diameter to ˜1 cm, and the power consumption is reduced from ˜100 W to ˜10 mW. Compared with commonly used electrical stimulators such as intra-cortical stimulators, MCMS does not require penetration of the dura, resulting in significantly lower risks and invasiveness.

In one or more embodiments, the above features are mainly enabled by (1) a new concept of integrating a magnetic stimulator in a skull burr hole, (2) an optimization flow to determine the optimal coil geometry while ensuring effective neural stimulations under size constraints, and (3) a custom-designed CMOS driver chip allowing for extremely high energy efficiency, as explained below. The stimulation efficacy is validated through transcranial stimulation using mice.

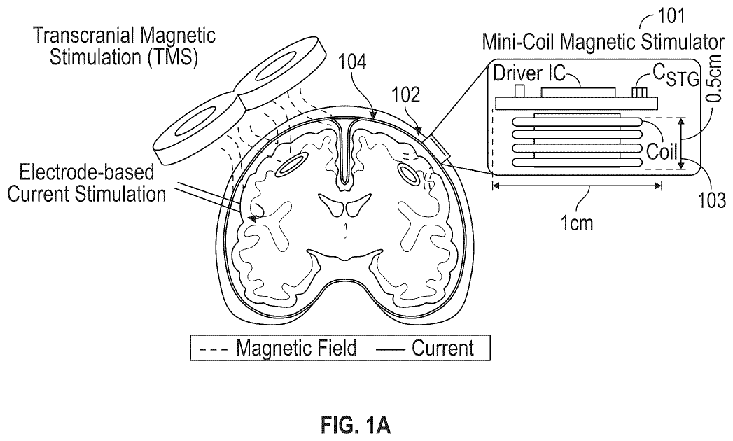

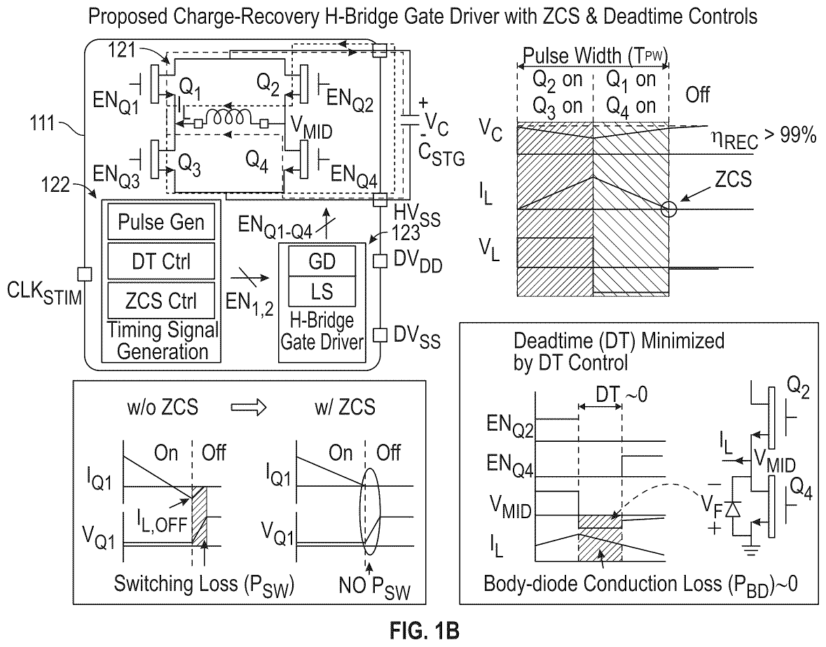

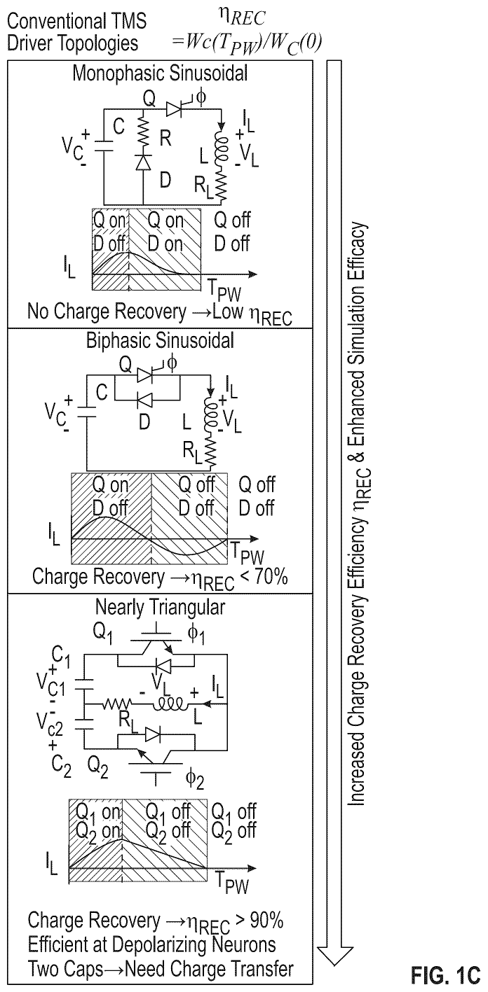

FIGS. 1A-1C illustrate the proposed mini-coil magnetic stimulator (MCMS) sealed within a burr hole for cortex stimulation. The MCMS uses substantially triangular waveforms and integrates an H-bridge gate driver with ZCS and deadtime controls to maximize energy efficiency.

Specifically, FIG. 1A compares three neural stimulation methods: Transcranial Magnetic Stimulation (TMS), Electrode-Based Stimulation, and the proposed MCMS.

TMS is non-invasive but requires bulky coils (10-20 cm) and high operating currents, limiting portability and spatial resolution. Electrode-based stimulation provides precision but risks glial scar formation and electrode encapsulation due to direct tissue contact.

The MCMS 101 overcomes these challenges with a compact design implanted in a small skull burr hole 102 (1.4 cm diameter) above the dura 104, thereby avoiding tissue contact. The MCMS's 1 cm coil 103 and integrated driver are energy-efficient and less invasive, which offers safer and more localized neural stimulation compared to traditional methods. In one or more embodiments, the coil 103 in the MCMS 101 may have a diameter of 1 cm or less, and in some cases, up to 1.4 cm.

Embodiments of the MCMS 101 may be hermetically sealed within a skull burr hole 102 for human brain cortex stimulation. This allows for point-of-care stimulation with greatly improved spatial resolution and >10,000×DC power reduction compared to TMS. Moreover, MCMS 101 sits in the skull and does not penetrate through the dura 104, which imposes considerably lower risk and invasiveness than implanting intracortical electrodes or micro-coils into the brain tissue.

FIG. 1B illustrates the operation of a charge-recovery coil driver circuit 111 with integrated zero-current switching (ZCS) and deadtime (DT) control mechanisms, designed to maximize efficiency in the MCMS 101.

At the top, the coil driver circuit 111 circuit is shown, which includes the H-bridge power stage 121 that includes four switches (Q₁-Q₄) arranged in an “H” configuration. The circuit includes a storage capacitor (C_{STG}) that powers the coil (L) during magnetic stimulation. The timing signal generation module 122 controls the operation of the H-bridge using a pulse generator, deadtime (DT) controller, and a ZCS controller. The pulse width (T_{PW}) is programmable, and the transitions between switching phases (e.g., Q₂/Q₃ turning off and Q₁/Q₄ turning on) are synchronized to ensure efficient charge recovery, which achieves >99% charge recovery efficiency (η_{REC}).

More specifically, in one or more embodiments, the coil driver circuit 111 may further include a first pulse generator configured to convert a ramp signal into a first short pulse, a deadtime (DT) controller that generates a first timing control signal and determines the rising edge of a second timing control signal based on the first short pulse, a second pulse generator that outputs a second short pulse in response to the rising edge of the second timing control signal, and a zero-current switching (ZCS) controller that produces the falling edge of the second timing control signal based on the first timing control signal and the second short pulse. The first and second timing control signals collectively control the activation and deactivation of the plurality of switches in the H-bridge power stage 121 of the coil driver circuit 111.

The impact of ZCS is also illustrated in FIG. 1B. Without ZCS, switching losses (P_{SW}) may occur when switches turn off with non-zero current (I_{Q1} or I_{L_OFF}). By enabling ZCS, the switches turn off exactly at zero current, thereby eliminating P_{SW} and improving power efficiency.

FIG. 1B further focuses on DT control, which avoids the overlap of switch transitions to prevent shoot-through currents and minimizes the dead time between transitions to reduce body-diode conduction loss (P_{BD}). DT is precisely controlled using a delay-locked loop (DLL) to achieve near-zero DT. As shown in FIG. 1B, the body-diode conduction current (through Q₂ and Q₄) is minimized.

More specifically, as shown in FIG. 1B, the coil driver circuit 111 includes an H-bridge power stage 121, where the switches are arranged in an “H” configuration to control the direction of current through the coil. The coil driver circuit 111 also incorporates an off-chip storage capacitor to support power stability. The H-bridge gate driver 123 includes a gate driver (GD) and a low-side driver (LS) that manage the switching of the transistors. Additionally, the coil driver circuit 111 may include a timing signal generation module 122 that provides control signals, including a pulse generator, a deadtime controller, and a zero-current switching controller.

FIG. 1C illustrates and compares three types of driver topologies used in transcranial magnetic stimulation (TMS). Specifically, FIG. 1C focuses on the η_{REC} and waveform characteristics. Among these three, the substantially triangular waveform has the highest charge recovery efficiency (η_{REC}) and requires the lowest energy for depolarizing neurons.

As shown in FIG. 1C, the monophasic sinusoidal driver employs a simple design with a single capacitor (C), diode, and resistor to generate a unidirectional sinusoidal current (I_{L}). However, this topology lacks any charge recovery mechanism, resulting in low efficiency (η_{REC}<50%) and significant energy loss. This design is less effective for prolonged use and less efficient in delivering stimulation to neural tissue.

The biphasic sinusoidal driver improves upon the monophasic design by incorporating a second capacitor (C₂), enabling partial charge recovery. This design produces a bidirectional sinusoidal current, which enhances neural stimulation efficacy compared to the monophasic waveform. However, its charge recovery efficiency remains limited to η_{REC}<70%, making it moderately efficient for energy usage.

The substantially triangular driver is based on the charge transfer between the two storage capacitors (C₁, C₂) with switches (Q₁, Q₂) to generate a substantially triangular waveform. This design achieves significantly higher efficiency (η_{REC}>90%) due to improved charge recovery mechanisms. The triangular waveform is more effective at depolarizing neurons, making it optimal for neural stimulation. However, it requires more complex charge transfer between two large capacitors, increasing design complexity and size.

Overall, the progression from monophasic to triangular waveforms demonstrates increasing energy efficiency and improved neural stimulation performance, with the substantially triangular waveform offering the best balance of efficacy and efficiency.

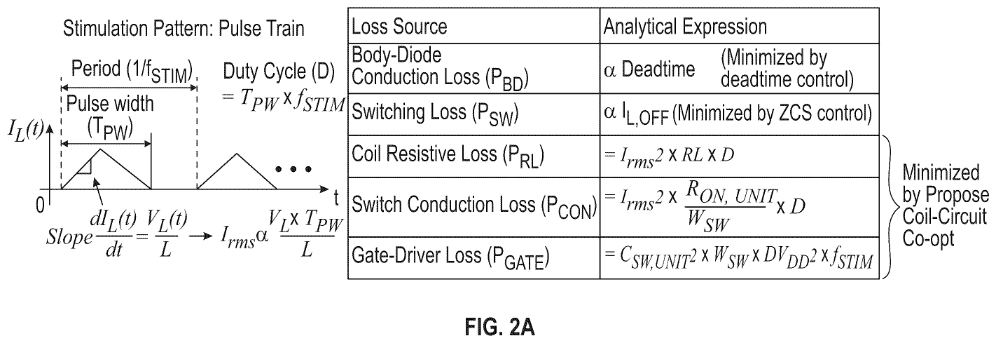

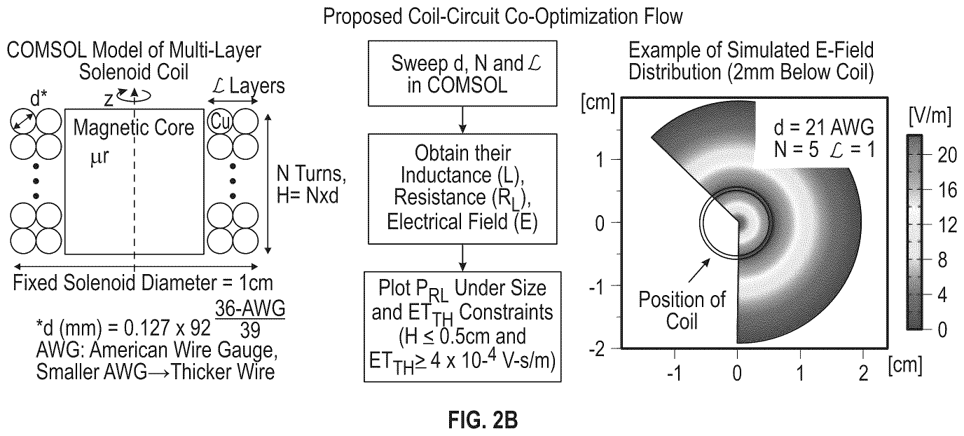

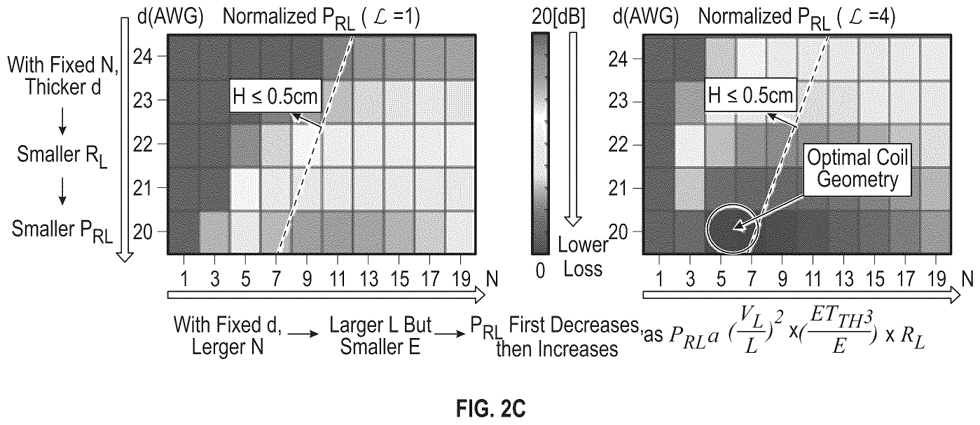

FIGS. 2A-2C show a coil-circuit co-optimization flow that determines the optimal coil geometry and transistor size of the coil driver circuit 111 to minimize losses and maximize energy efficiency.

Specifically, FIG. 2A describes a pulse train stimulation pattern and key power loss sources, along with strategies to minimize them. The duty cycle (D) is determined by the pulse width (T_{PW}) and frequency (f_{STIM}), while the coil current (I_{L(t)}) rises linearly based on coil voltage (V_{L}) and inductance (L).

Key loss sources include body-diode conduction loss (P_{BD}), minimized by deadtime control, and switching loss (P_{SW}), reduced through zero-current switching (ZCS). Coil resistive loss (P_{RL}) and switch conduction loss (P_{CON}) are addressed via coil-circuit co-optimization and transistor sizing, respectively. Gate-driver loss (P_{GATE}) is mitigated by optimizing gate driver design. To optimize neural stimulation, the stimulation threshold (ET_{TH}) is defined as the product of the electric field (E) and pulse width (T_{PW}), based on the integrate-and-fire model. A COMSOL parametric sweep varies coil geometry to minimize resistive power loss (P_{RL}) under size and ET_{TH} constraints. The H-bridge transistor width (W_{SW}) is then optimized to minimize P_{CON}+P_{GATE} while supporting the required coil current.

FIG. 2B details the coil-circuit co-optimization process for the Mini-Coil Magnetic Stimulator (MCMS), using the COMSOL simulation to optimize coil geometry based on performance metrics.

According to one or more embodiments, the COMSOL model represents a multi-layer solenoid coil 103 with a fixed diameter of 1 cm. Parameters such as wire diameter (d), number of turns (N), and layers ( [symbol] ) are adjusted to achieve the desired inductance (L), resistance (R_{L}), and electric field (E). The wire thickness is defined by the American Wire Gauge (AWG), with smaller AWG indicating thicker wire. In one or more embodiments, the coil 103 can be designed in shapes other than solenoid, such as a tapered-tip shape.

The flowchart outlines the process, starting with sweeping parameters wire diameter (d), number of turns (N), and layers ( [symbol] ) in a COMSOL simulation. These parameters are optimized to obtain the coil's inductance (L), resistance (R_{L}), and electric field (E). The results are then used to plot resistive power loss (P_{RL}) while ensuring the design adheres to size (H≤0.5 cm) and stimulation threshold constraints (E_{TH}≥4×10⁻⁴ V·s/m). In other words, parameters of the coil 103 including number of layers, inductance, resistance, and electric field may be determined based on a coil-circuit co-optimization flow. This optimization flow identifies coil parameters that minimize power loss while maintaining effective neural stimulation, thereby ensuring efficient and compact design.

According to one or more embodiments, FIG. 2B further shows an example E-field distribution (2 mm below the coil) of how the electric field strength varies with geometry. This is critical for meeting the stimulation threshold (E_{TH}) while maintaining compact dimensions.

FIG. 2C show plots illustrate the optimization process. In the left plot ( [symbol] =1), increasing wire thickness (d) reduces coil resistance (R_{L}), minimizing coil resistive loss (P_{RL}). In the right plot ( [symbol] =4), _{PRL} decreases initially with increasing turns (N) but then rises due to a reduced electric field (E). The optimal geometry balances P_{RL} while satisfying constraints.

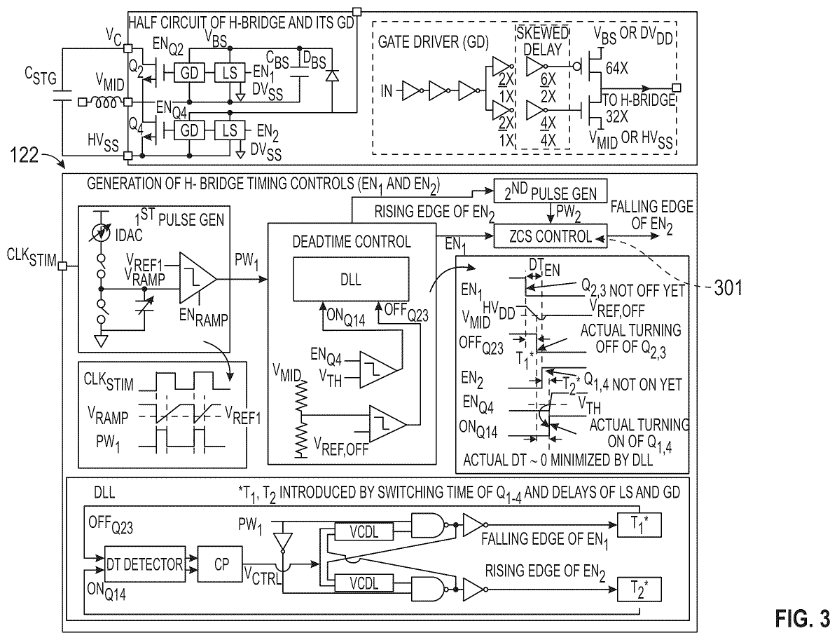

FIG. 3 is a schematic drawing of half circuit of the H-bridge gate driver 123 and its gate driver, generation of H-bridge timing controls (EN₁ and EN₂), and the detailed operation of the closed-loop deadtime control to minimize P_{BD}.

In one or more embodiments, the GD may employ skewed delays to prevent shoot-through in the last stage. The GD input may be connected to the H-bridge timing controls (EN₁ or EN₂) through a level shifter (LS). LS is implemented using a current-mode trigger-based topology to achieve a low propagation delay. In one or more embodiments, a bootstrap circuit, including a capacitor C_{BS} and a diode D_{BS}, may be used to provide the supply for the high-side LS and GD. In one or more embodiments, the bootstrap circuit provides voltage up to 24V. In one or more embodiments, the voltage can exceed 24V depending on the transistors used. By utilizing a different type of transistor, the voltage can even reach up to 70V.

FIG. 3 further illustrates the generation of EN₁ and EN₂, including two pulse generators, a DT controller, and a ZCS controller 301 in the timing signal generation module 122. Each pulse generator includes a charge-pump-based ramp generator and a comparator, offering a wide tuning range for the pulse width (T_{PW}) for various stimulation targets. In the first pulse generator, its ramp signal V_{RAMP} is derived from an external clock CLK_{STIM}, which defines the pulse repetition frequency. The pulse generator converts a ramp signal into a short pulse. V_{RAMP}is converted to a short pulse PW₁ by comparing it with the comparator's reference voltage (V_{REF1}). PW₁ is then routed to the DT controller, which outputs EN₁ and the rising edge of EN₂. The falling edge of EN₂ is generated by the second pulse generator followed by the ZCS controller 301. More specifically, in one or more embodiments, the DT controller may monitor turn-on and turn-off events in the H-bridge gate driver 123 and adjusts a control voltage in a voltage-controlled delay line (VCDL) to maintain a substantially zero actual deadtime.

In one or more embodiments, at least one of the two pulse generator may include a charge-pump-based ramp generator that generates a programmable ramp signal that defines a pulse width and duration; compares a ramp signal against a threshold; and adjusts an activation timing of the H-bridges switches.

In addition, according to one or more embodiments, the time interval between the falling edge of EN₁ and the rising edge of EN₂ (i.e., DT_{EN}) does not reflect the actual DT, due to the switching time of Q₁-Q₄ and the delay introduced by the LS and GD. To accurately measure the DT, two indicators are introduced: OFF_{Q23} and ON_{Q14}. Specifically, OFF_{Q23} is triggered when the source terminal of Q₂ (i.e., V_{MID}) drops from HV_{DD} to a predefined threshold voltage (1V by default), indicating the actual turning off of switch Q₂/Q₃; ON_{Q14} is triggered when Q₄'s gate voltage (EN Q₄) rises above V_{TH}, indicating the actual turning on of switch Q₁/Q₄. OFF_{Q23} and ON_{Q14} are synchronized using a delay-locked loop (DLL), achieving substantially 0 actual DT in a closed-loop fashion. The voltage-controlled delay line (VCDL) then defines the non-overlapping time between EN₁and EN₂ (i.e., DT_{EN}).

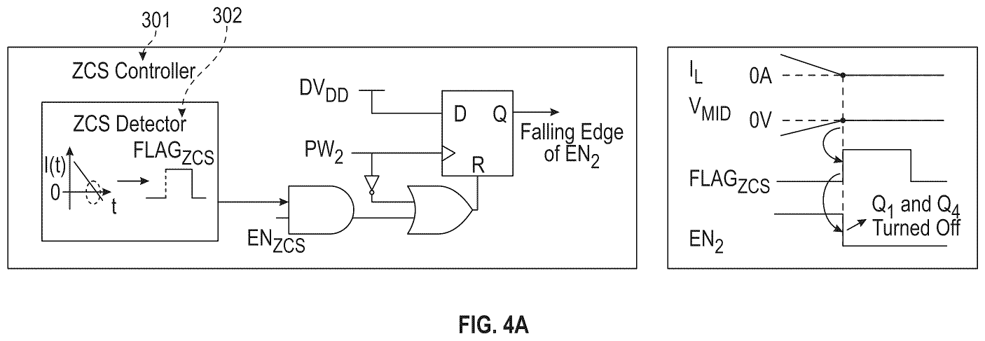

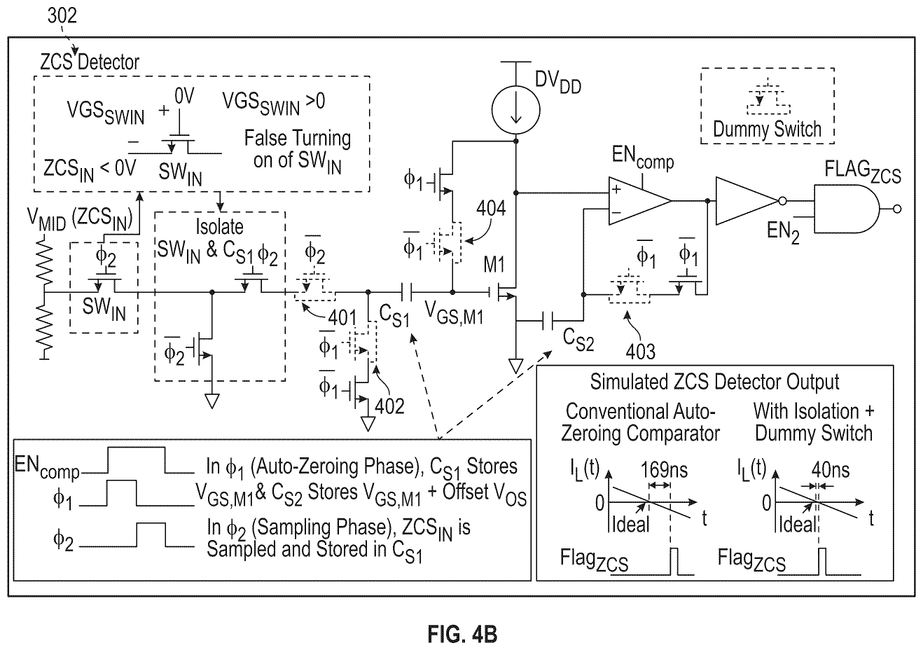

FIGS. 4A and 4B are schematic drawings of a timing diagram of the ZCS controller 301. The ZCS detection accuracy is greatly improved by including a set of isolation switches and dummy switches 401-404.

In one or more embodiments, the ZCS controller 301 may include a control circuit that detects zero-current points in a switching cycle and triggers the plurality of switches to turn off at these points. In one or more embodiments, the ZCS controller 301 may further include an auto-zeroing comparator that enables ZCS detection under process, voltage, and temperature variations. The ZCS controller 301 may further include one or more isolation switches and one or more dummy switches.

As shown in FIG. 4A, in one or more embodiments, for the ZCS detector 302, when I_{L}(t) drops to 0, FLAG_{ZCS} turns high, which resets the flip-flop and generates the falling edge of EN₂. This falling edge subsequently turns off Q₁ and Q₄.

FIG. 4B further explains the design and operation of the ZCS detector 302 used in the coil driver circuit 111. The voltage across Q₄ (i.e., V_{MID}) serves as the ZCS detector input, which transitions from negative to positive when I_{L}(t) becomes 0.

The ZCS detector 302 operates in two phases. In the auto-zeroing phase (Φ₁), capacitors C_{S1} and C_{S2} store the gate-source voltage (V_{GS,M1}) of transistor M1, along with any offset voltage (V_{OS}). During the sampling phase (Φ₂), the zero-crossing signal (ZCS_{IN}) is sampled and stored in C_{S1}, allowing the circuit to determine if the coil current has reached zero. A comparator processes this signal and triggers the FLAG_{ZCS} output, which controls switch timing.

According to one or more embodiments, isolation switches prevent unwanted interactions between S_{WIN} and C_{S1}, which avoids false signals during the sampling phase. Dummy switches 401-404 reduce charge injection and clock feedthrough effects, ensuring more precise signal sampling and minimal timing deviation.

In one or more embodiments, dummy switches 401-404 around C_{S1} and C_{S2} may be incorporated to shield them from undesired charge injection when the sampling switches are turned off. The improved design significantly enhances performance. A conventional ZCS detector without isolation and dummy switches 401-404 shows a deviation of 169 ns from the ideal zero-crossing point. In contrast, the enhanced design reduces this deviation to just 40 ns, closely aligning with the ideal timing. In one or more embodiments, the ZCS controller 301 may include at least four dummy switches to improve sensing accuracy of zero-crossing events.

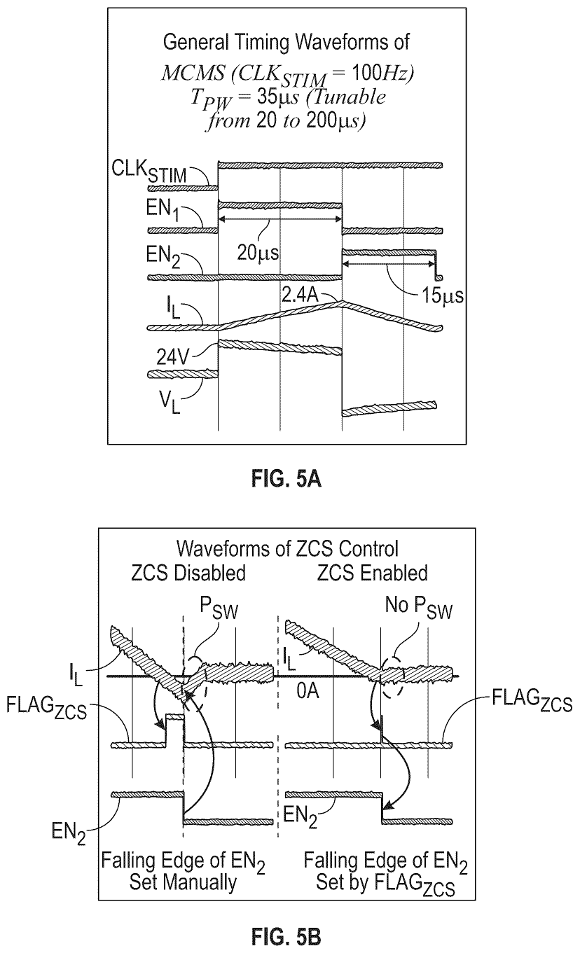

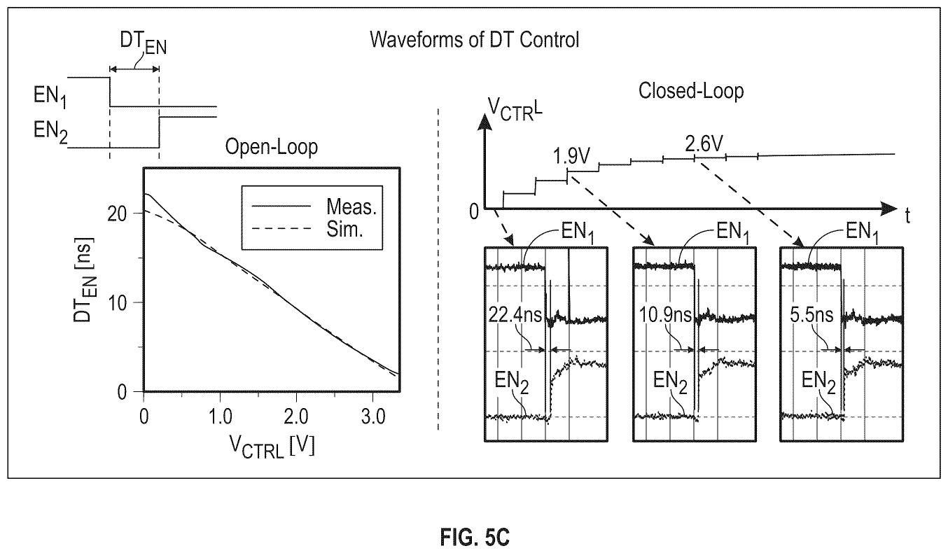

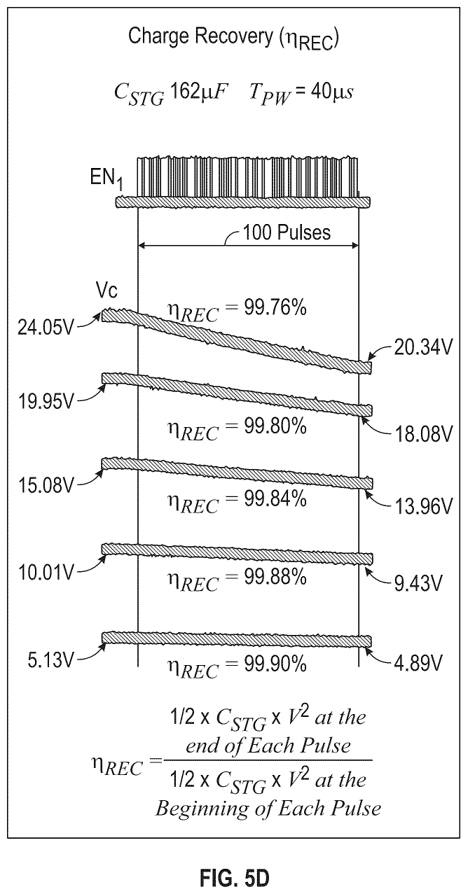

FIGS. 5A-5D show measured waveforms of MCMS 101, its ZCS controller, and its deadtime controller. A consistent charge recovery efficiency (η_{REC}) of >99% is achieved across a wide range of C_{STG}'s initial voltage (5-24V). In one or more embodiments, a substantially triangular waveform is achieved for I_{L}(t) with programmable pulse widths for EN₁ and EN₂. For the ZCS control, FLAG_{ZCS} is triggered when I_{L}(t) drops to 0, demonstrating the desired operation of the ZCS detector 302. Additionally, when enabling the ZCS control, I_{L}(t) remains 0, thus avoiding P_{SW}. For the DT control, the open-loop behavior may be measured by monitoring DT_{EN} VS. VCDL's control voltage (V_{CTRL}), which closely aligns with the simulation. Then, the settling behavior is measured with close loop. VCTRL takes ˜8 cycles to reach the steady state with DT_{EN}=5.5 ns. Finally, the η_{REC} is measured with a 162 μF C_{STG}. The voltage stored in C_{STG} after 100 stimulation pulses remains >84.6% of its initial voltage.

Specifically, FIG. 5A illustrates the general timing waveforms for the MCMS 101 operating at a clock frequency (CLK_{STIM}) of 100 Hz. The pulse width (T_{PW}) is set to 35 μs but is adjustable within a tunable range of 20 to 200 μs. Key control signals, EN1 and EN2, manage the activation of the H-bridge switches, and enable precise timing of the coil operation. During the stimulation phase, the coil current (I_{L}) ramps up to 2.4 A, while the coil voltage (V) starts at 24 V and decreases as energy is transferred to the coil 103.

FIG. 5B compares the waveforms of the Zero-Current Switching (ZCS) control in two scenarios: ZCS disabled and ZCS enabled. When ZCS is disabled, the falling edge of EN₂ is set manually, leading to switching loss (P_{SW}) as the coil current (I_{L}) does not reach zero before switching. In contrast, with ZCS enabled, the falling edge of EN₂ is automatically controlled by the FLAG_{ZCS} signal, ensuring that switching occurs precisely when I_{L} reaches zero. This eliminates switching loss of the circuit.

FIG. 5C illustrates the waveforms of DT control in both open-loop and closed-loop configurations. In the open-loop mode, the deadtime (DT_{EN}) between the signals EN₁ and EN₂ is shown as a function of the control voltage (V_{CTRL}), with simulated and measured results closely matching. In the closed-loop mode, DT_{EN}is dynamically adjusted, starting at 22.4 ns for V_{CTRL}=1.9V and decreasing to 10.9 ns and eventually to 5.5 ns as V_{CTRL} stabilizes at 2.6 V. This closed-loop operation minimizes the deadtime, ensuring efficient transitions between switching events while preventing overlap, thereby reducing power losses and improving system performance.

FIG. 5D The image illustrates the charge recovery efficiency (η_{REC}) of the system over 100 pulses with a storage capacitor (C_{STG}=162 μF) and a pulse width (T_{PW}=40 μs). Starting with an initial voltage (V_{C}) of 24.05 V, the capacitor's voltage gradually decreases after each pulse but maintains high charge recovery efficiencies at each step. The efficiency remains consistently above 99%, with the final voltage at 20.34 V after 100 pulses. The formula for η_{REC} compares the energy retained (½·C_{STG}·V²) at the end of each pulse to the energy at the beginning.

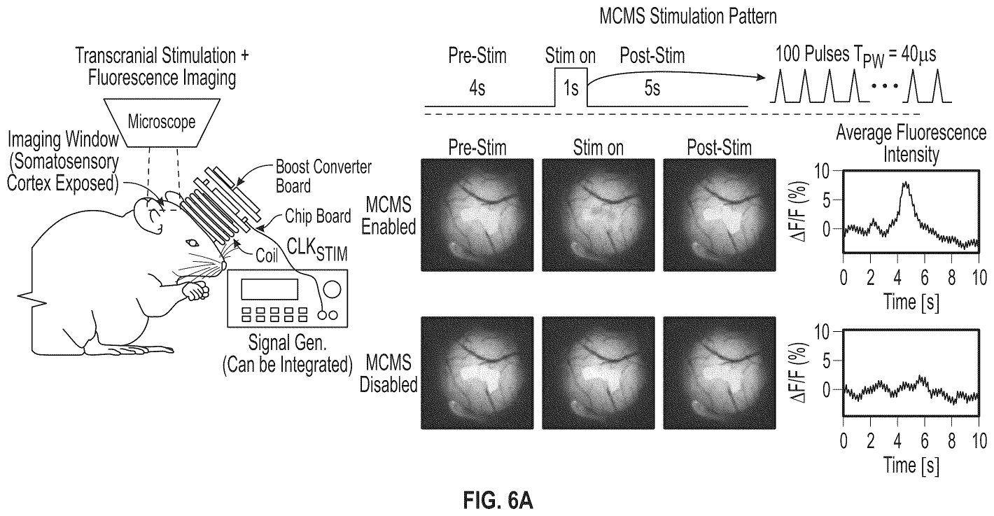

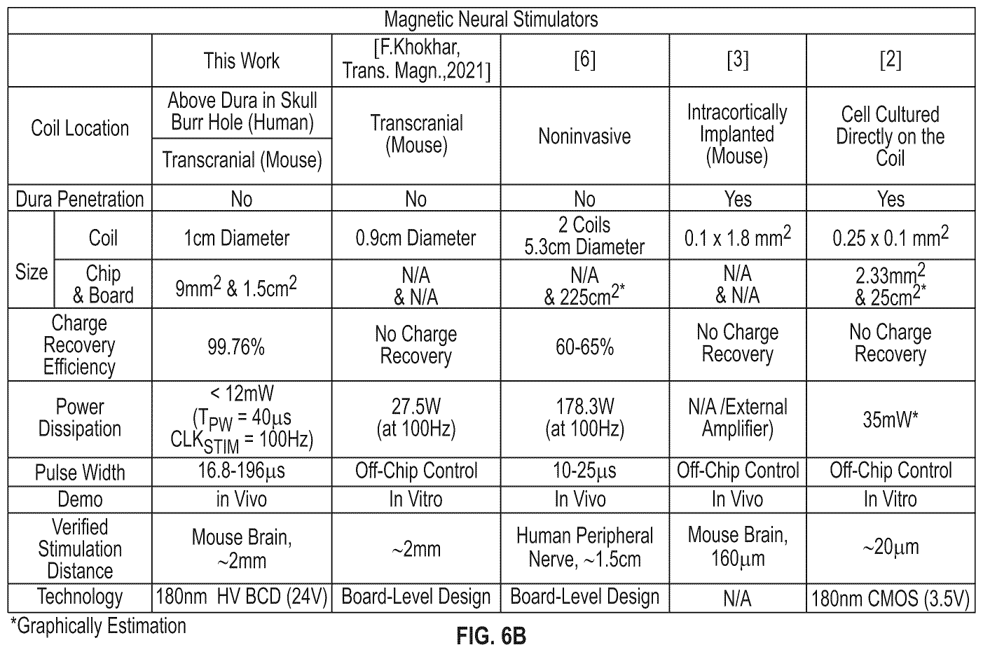

FIG. 6A-6B describe an experimental setup of in vivo stimulation, measured fluorescence increase in response to MCMS 101 (with a p-value of ˜10⁻⁸ under T-test), and a comparison with other magnetic neural stimulators.

The MCMS device's stimulation capability is validated in vivo. As shown in FIG. 6A, in the experiment, the mouse brain was stimulated transcranially and the brain activity was recorded using wide-field Ca²⁺ fluorescence imaging. To minimize the impact of spontaneous neural activity, the fluorescence intensity is averaged across 60 trials. Each trial includes 4 s of pre-stimulation, 1 s of stimulation, and 5 s of post-stimulation. When the MCMS 101 is activated, a significant fluorescence increase is observed in the somatosensory cortex, demonstrating effective neural stimulation.

Compared with prior reported magnetic neural stimulators, as shown in FIG. 6B, the MCMS 101 achieves the highest η_{REC} and the lowest DC power, thanks to the power loss reduction enabled by the charge-recovery coil driver circuit 111, its ZCS and DT controls, and the proposed coil-circuit co-optimization flow. Additionally, the MCMS 101 stays above the dura 104, making it less invasive and posing a lower risk than intracortical electrodes and micro-coils that require penetration of the dura 104. In summary, this work demonstrates the great potential of the proposed MCMS 101 as an attractive alternative to existing intracortical implants.

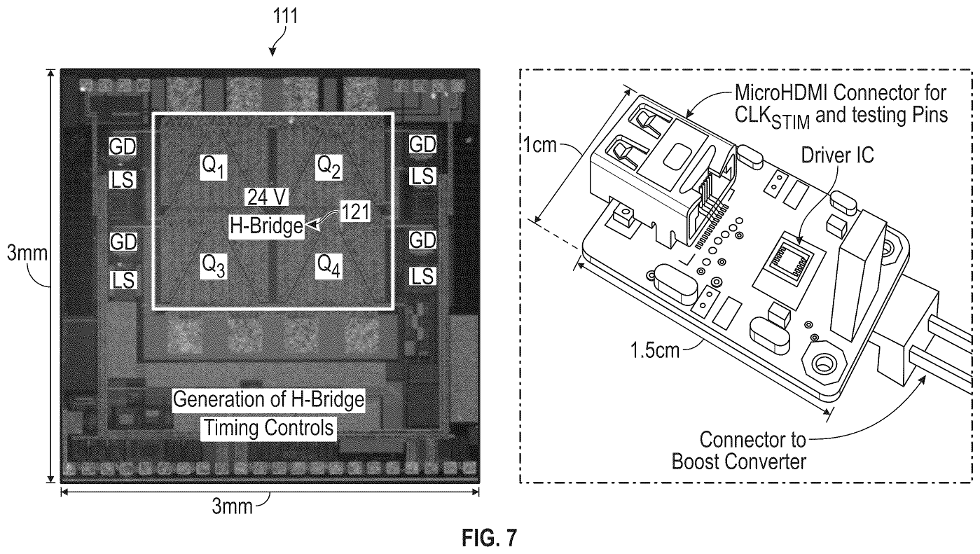

FIG. 7 is a die micrograph and a picture of the testing PCB. In one or more embodiments, the die micrograph of the coil driver circuit 111 integrated circuit may be 3 mm×3 mm in size. As described above and shown in FIG. 7, the H-bridge power stage 121 includes four switches (Q₁-Q₄) to control the current flow in the coil. In one or more embodiments, the circuit operates at 24V and integrates essential timing controls for precise stimulation waveforms. GD and LS are strategically placed to ensure efficient signal transmission and switching.

In the photograph of the testing PCB containing the driver IC, the board is compact, measuring 1.5 cm×1 cm, and includes a micro HDMI connector for signal input (e.g., CLK_{STIM}) and testing pins. Notably, the board can be further miniaturized, as its current size is for testing purposes. The board is also equipped with a connector to the boost converter, which provides the required power to the circuit by allowing the input to come from a battery rather than being limited to benchtop high-voltage power supplies.

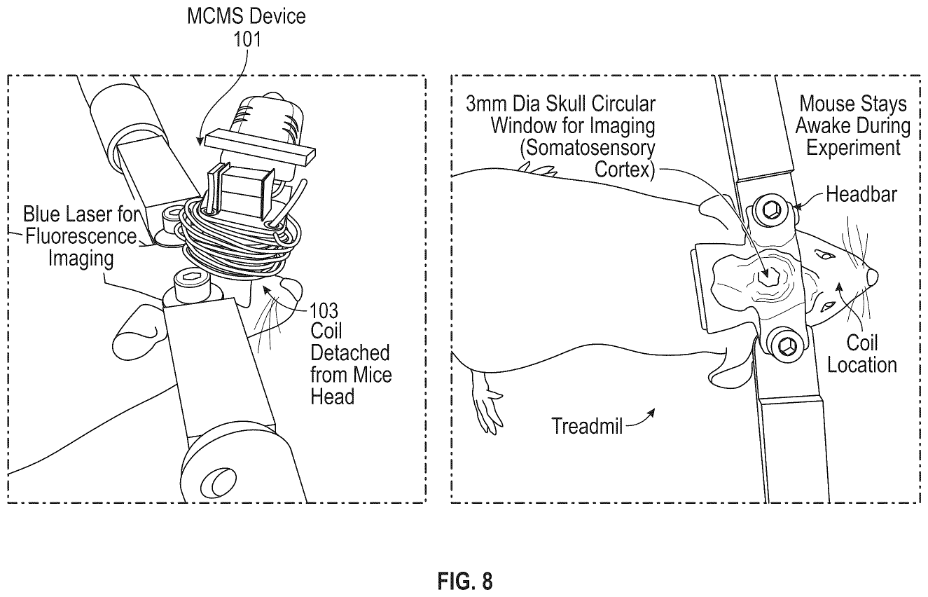

FIG. 8 illustrates an experiment setup of in vivo transcranial stimulation. The mouse first went through a survival surgery to drill a 3 mm-diameter circular craniotomy over its somatosensory cortex, so that its brain activity can be monitored using wide-field Ca²⁺ fluorescence imaging. The stimulation experiment is carried out by placing the mouse on a treadmill with a fixed head position. The stimulation coil 103 is placed transcranially and remains above the forehead, ensuring the observed responses are not due to any potential mechanical vibrations. Additionally, a white noise of ˜70 dB is applied to eliminate potential auditory interference.

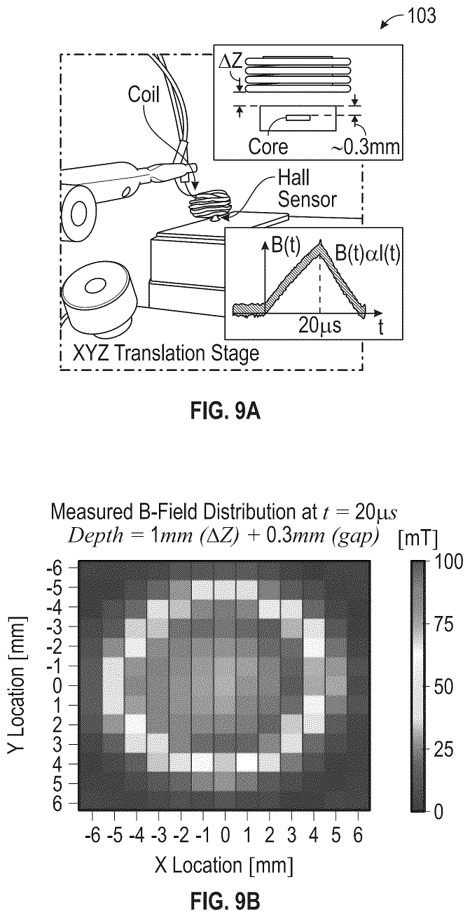

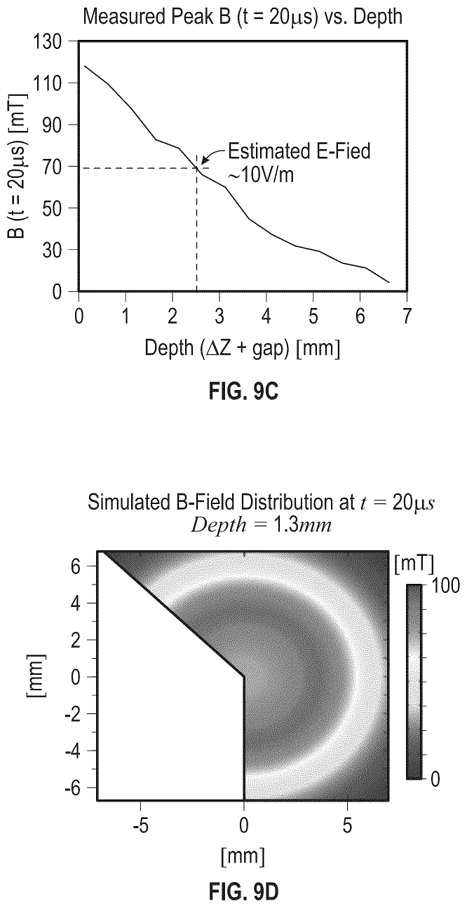

FIGS. 9A-9D show a measurement result of the magnetic (B) field distribution at a distance (ΔZ) of 1 mm below the coil 103 using a hall sensor (A1324LUA-T). Specifically, in FIG. 9A, considering the 0.3 mm gap between the top of the hall sensor and its core, the testing data (top right) actually represents the B field at a depth of 1.3 mm. Further, as shown in FIG. 9B, the measured B-field distribution and its peak value (˜100 mT at t=20 μs) are comparable to the COMSOL simulations. Further, as shown in FIGS. 9C and 9D, the peak B vs. ΔZ also has been measured, showing a working depth of ˜2.5 mm.

Although the disclosure has been described with respect to only a limited number of embodiments, those skilled in the art, having benefit of this disclosure will appreciate that various other embodiments may be devised without departing from the scope of the present invention. Accordingly, the scope of the invention should be limited only by the attached claims.

What is claimed is:

1. A mini-coil magnetic neural stimulator (MCMS) comprising:
a coil that generates magnetic stimulation; and
a coil driver circuit that controls the magnetic stimulation,
wherein the MCMS is implanted in a skull burr hole without penetrating a dura of a patient, and
wherein no external leads are connected to the MCMS through a scalp of the patient.

2. The MCMS according to claim 1, wherein the coil driver circuit comprises:
an H-bridge power stage that comprises:
a plurality of switches that are arranged in a shape of letter “H;” and
a storage capacitor that stores energy that is used to power the coil during the magnetic stimulation;
an H-bridge gate driver that comprises:
a gate driver that controls switching of the plurality of switches; and
a timing signal generation module.

3. The MCMS according to claim 2, wherein the timing signal generation module that further comprises:
a first pulse generator that converts a ramp signal into a first short pulse;
a deadtime (DT) controller that outputs a first timing control signal and a rising edge of a second timing control signal based on the first short pulse;
a second pulse generator that outputs a second short pulse based on the rising edge of the second timing control signal; and
a zero-current switching (ZCS) controller that generates a falling edge of the second timing control signal based on the first timing control signal and the second short pulse,
wherein the first timing control signal and the second timing control signal control activation of the plurality of switches.

4. The MCMS according to claim 3, wherein the ZCS controller comprises a control circuit that detects zero-current points in a switching cycle and triggers the plurality of switches to turn off at these points.

5. The MCMS according to claim 3, wherein the ZCS controller further comprises an auto-zeroing comparator to enable ZCS detection under process, voltage, and temperature variations.

6. The MCMS according to claim 5, wherein the ZCS controller further comprises one or more isolation switches and one or more dummy switches to improve sensing accuracy of zero-crossing events.

7. The MCMS according to claim 6, wherein the ZCS controller comprises at least 4 dummy switches to minimize undesired charge injection.

8. The MCMS according to claim 3, wherein the DT controller monitors turn-on and turn-off events in the coil driver circuit and adjusts a control voltage in a voltage-controlled delay line (VCDL) to maintain a substantially zero actual deadtime.

9. The MCMS according to claim 3, wherein at least one of the first pulse generator and the second pulse generator comprises a charge-pump-based ramp generator that
generates a programmable ramp signal that defines a pulse width and duration;
compares a ramp signal against a threshold; and
adjusts an activation timing of the H-bridges switches.

10. The MCMS according to claim 2, wherein the gate driver further comprises:
a bootstrap circuit including a bootstrap capacitor and diode; and
a level shifter that is implemented using a current-mode trigger-based topology.

11. The MCMS according to claim 10, wherein the bootstrap circuit provides voltage up to 70V.

12. The MCMS according to claim 2, wherein a number of the plurality of switches is 4 switches.

13. The MCMS according to claim 1, wherein the magnetic stimulation applies a waveform that is substantially triangular.

14. The MCMS according to claim 2, wherein the coil generates a magnetic field that induces electric currents in a neural tissue.

15. The MCMS according to claim 1, wherein the coil has a diameter of less than 1.4 cm.

16. The MCMS according to claim 1, wherein parameters of the coil including number of layers, inductance, resistance, and electric field are determined based on a coil-circuit co-optimization flow.

17. The MCMS according to claim 16, wherein the layers are arranged in a solenoidal or tapered-tip configuration.

18. The MCMS according to claim 1, wherein the magnetic stimulation applies a waveform that is biphasic.

19. A method of treating neurological disorders, comprising:
implanting a mini-coil magnetic neural stimulator (MCMS) that comprises a coil for magnetic stimulation and a coil driver circuit in a skull burr hole without penetrating a dura of a patient;
wherein no external leads are connected to the MCMS through a scalp of the patient.

20. The method according to claim 19, wherein the coil driver circuit comprises:
an H-bridge power stage that comprises:
a plurality of switches that are arranged in a shape of letter “H;”
a storage capacitor that stores energy that is used to power the coil during the magnetic stimulation;
an H-bridge gate driver that comprises:
a gate driver that controls switching of the plurality of switches; and
a timing signal generation module.
